(19) United States
(12) Patent Application Publication
(10) Pub. No.: US 2012/0010499 A1
(43) Pub. Date: Jan. 12, 2012

(54) USE OF NANOPARTICLES FOR THE TREATMENT OF CANCER

(76) Inventors: Sam M. Janes, London (GB); Mark F. Lythgoe, London (GB); Quentin Pankhurst, London (GB)

(21) Appl. No.: 13/101,960

(22) Filed: May 5, 2011

Related U.S. Application Data
(60) Provisional application No. 61/332,556, filed on May 7, 2010.

(51) Int. Cl. A61M 37/00; A61B 5/00; A61N 5/00; A61K 35/48; A61P 35/00

(52) U.S. Cl. 600/411; 424/93.21; 600/1; 604/500

(57) ABSTRACT

The invention relates to the tracking of mesenchymal stem cells (MSCs) labeled with magnetic nanoparticles using magnetic resonance imaging (MRI) and the use of this method for the treatment of cancer.

### FIELD OF THE INVENTION

The invention relates to the tracking of mesenchymal stem cells (MSCs) labelled with metal or metal oxide nanoparticles imaging and the use of this method for the treatment of cancer.

### BACKGROUND OF THE INVENTION

The poor survival of both lung cancer patients and those with other forms of pulmonary metastatic disease relates partly to the inability to deliver locally targeted therapeutic agents. Recently, exogenous MSCs from the bone marrow compartment have been used to attenuate several carcinoma models (1-6, 26-28). In some of these studies, the MSCs, which are carrying anti-tumour therapies, have been delivered locally (2, 26-28). In other studies, the MSCs have been delivered systemically and migrate to the site of the tumour. Once the MSCs arrive at the site of the tumour, they contribute to tumour reduction (1, 3-6).

It has been shown previously in murine cancer studies that human MSCs expressing tumour necrosis factor-related apoptosis-inducing ligand (TRAIL) can provide targeted delivery of this pro-apoptotic agent to breast cancer metastases (1). Similarly, MSCs transduced to express IFNβ or the immunostimulatory chemokine CX3CL1 have also been shown to reduce tumour burden in murine glioma (3), breast (5), melanoma (4), and colorectal models (6) with an improvement in survival.

The ability of bone marrow-derived stem cells to migrate to areas of injury in a range of pathological conditions suggests that they may be ideal vectors for therapeutic delivery. MSCs possess a number of properties that make them suitable candidates. MSCs are easily obtained from a simple bone marrow-aspirate and are readily expanded in culture without losing their multi-lineage potential. They are readily transducible, allowing for simple ex vivo modification (7). Finally, they seem to be relatively non-immunogenic (8) due to their lack of MHC2 and co-stimulatory molecules CD80, CD86 and CD40 (9). This may allow the delivery of genetically dissimilar MSCs without the need for immunomodulation or subsequent immunosuppressive therapy for the recipient. Because of these properties, MSCs have considerable therapeutic potential in tumour therapy.

In a clinical setting, the use of MSCs to deliver therapies for the treatment of a tumour creates a need for an imaging tool which can be used to confirm targeted delivery of the therapy to the tumour. For clinical applications, the ability to track MSCs homing to primary tumours and metastases using a simple non-invasive scan would be of great benefit. Although murine models have shown a lot of promise for transduced MSCs in cancer therapy, many uncertainties still remain. The ability to systemically visualize the therapy and the response of the tumour will allow for more informed decisions about the optimum timing of MSC therapy, as well as the number of treatments required.

Recently, imaging contrast agents have emerged that open up the possibility of visualizing stem cell transplants in vivo using MRI. Superparamagnetic iron oxide (SPIO) (SPIO; Fe₃O₄) nanoparticles have been used to track engrafted cells in a variety of tissues (10) as well as targeted cell delivery (11). The nanoparticles generate a local magnetic field perturbation, which leads to a marked shortening of the MRI parameter T2. This is exhibited as hypointensity on magnetic resonance images, leading to the possibility of imaging the localization of these particles (10, 12).

The present invention uses this imaging technique to provide a tool for the tracking of MSCs homing towards tumour sites. MSCs labelled with magnetic nanoparticles can be detected in real time in vivo using MRI.

This imaging technique allows for confirmation of targeted delivery of anti-tumour agents that have been transported by the MSCs as well as enabling more informed decisions to be made regarding the optimal timing of MSC therapy. For instance, (1) used MSCs to deliver TRAIL to the site of a tumour. The expression of TRAIL, in this particular study, was sensitively controlled by doxycyline via an inducible lentivirus. The ability to detect the proximity of the transduced MSCs to the tumours with MRI could help define the optimal time window for the induction of TRAIL expression by detection of MSCs in the lung and any regression of the metastases.

The non-invasive tracking of MSCs has previously been studied with the use of bioluminescence (22) and whole-body micropositron emission tomography (23) with MSCs labelled with firefly luciferase or transduced to express HSV1-TK, respectively. However, the use of SPIO particles has the advantage of labelling MSCs without transduction but with the use of agents and facilities that are now frequently used in medical practice, thus providing direct clinical applicability. In the present invention, the nanoparticles had no adverse effect on the MSC differentiation, migration, survival and proliferation capacity.

Previous groups have studied the use of nanoparticles for detecting MSCs in vivo with direct injection into a cardiac scar (18) and direct injection into the brain (24). MSCs have also been tracked after intravenous injection in a Kaposi's sarcoma model (25). However, the present invention is the first assessment of the delivery of SPIO nanoparticle-labelled MSCs transporting anti-tumour agents to the tumour site for the treatment of the tumour.

The use of nanoparticles for the treatment of diseases using thermotherapy has been described (29-39). However, the present invention provides a mechanism for tracking the delivery of nanoparticles, which are contained within MSCs, to a tumour site using MRI.

### SUMMARY OF THE INVENTION

We have developed a novel technique which uses MRI to track the fate of magnetic nanoparticle labelled MSCs homing towards tumours. We have shown that the introduction of biocompatible iron oxide nanoparticles into MSCs enables localized cellular-level sensing while retaining full viability of the MSCs. This technique can be used to assess and manipulate the delivery of anti-tumour factors such as TRAIL to tumour sites. Also, the iron oxide nanoparticles contained within the MSCs can be used to kill the cells of the tumour by thermotherapy. This technique is useful, not only to track the delivery of MSCs which are directly injected at the site of a tumour, but also for the tracking of systemically delivered MSCs which home towards metastatic tumours, in particular pulmonary metastases.

The invention therefore provides a method of treating a tumour in a subject comprising the steps of:
delivering mesenchymal stem cells (MSCs) labelled with metal or metal oxide nanoparticles;
using imaging to detect homing of said MSCs containing said nanoparticles towards the cells of the tumour; and
killing said tumour cells by delivery of a pro-apoptotic factor by said MSCs to said tumour cells.

The invention also provides a method of treating a tumour in a subject comprising the steps of:
delivering MSCs labelled with magnetic nanoparticles;
detecting homing of said MSCs towards the cells of the tumour using MRI; and
killing said tumour cells by thermotherapy.

The invention also provides a method of treating pulmonary metastases comprising the steps of:
systemically delivering MSCs labelled with superparamagnetic iron oxide nanoparticles;
detecting homing of said MSCs towards the cells of said pulmonary metastases using MRI; and
killing said tumour cells by delivery of TRAIL to said tumour cells using a lentiviral vector within said MSCs.

The invention also provides a method of treating pulmonary metastases comprising the steps of:
systemically delivering MSCs labelled with superparamagnetic iron oxide nanoparticles;
detecting homing of said MSCs towards the cells of said pulmonary metastases using MRI; and
killing said tumour cells by thermotherapy.

### BRIEF DESCRIPTION OF THE DRAWINGS

FIG. 1: MSCs take up superparamagnetic iron oxide (SPIO) nanoparticles without affecting their phenotype.

A) (i) MSCs in culture (scale bar 20 μm), (ii) Prussian blue staining of MSCs after 24 hours of culture with SPIO nanoparticles (scale bar 20 μm), (iii) EM image showing cytoplasmic location of SPIO nanoparticles (scale bar 1 μm), (iv) Confocal image showing co-localization of SPIO nanoparticles (yellow), DiI (red) and the nuclear counterstain DAPI (blue) (scale bar 3 μm), (v) Differentiation to osteoblasts, Alizarin Red S staining (scale bar 40 μm), (vi) Differentiation to adipocytes, Oil-Red-O staining (scale bar 5 μm). B) (i) MSCs stained on the underside of the transwell membrane (scale bar 50 μm at ×4 mag, 10 μm at ×20 mag). (ii) The SPIO-loaded MSCs (Fe) migrate towards MDAMB231 breast cancer cells through a transwell membrane at the same rate as non-labeled MSCs (non Fe). C) The SPIO-loaded MSCs proliferate at the same rate as control MSCs. D) There is no increase in death and apoptosis of the SPIO-loaded MSCs compared to the non-labeled MSCs.

FIG. 2: Superparamagnetic iron oxide (SPIO)-loaded MSCs can be visualized by MRI in tumours at low concentrations.

A) 2×10⁶MDAMB231 cells were coinjected with i) 1×10⁵, ii) 1×10⁴, iii) 1×10³, iv) 100 SPIO-loaded MSCs, and visualized using a 9.4T MRI scanner 28 days later. The subcutaneous tumors can be seen in all mice (asterix). SPIO-loaded MSCs are visualized (arrow) when as few as 1×10³ cells were originally injected. There were no hypointesities on MRI with v) 1×10⁴ dead SPIO-loaded MSCs, or yl) free iron particle injection (100 ng) (n=2 in all groups). B) Prussian blue histochemistry (i,iii) and DiI (red) immunofluoresence (with DAPI nuclear counterstain—blue) (ii,iv) corresponding to the coinjection of 1×10⁵ (i-ii) and 1×10³ (iii-iv) SPIO-loaded MSCs confirming the iron stain colocalizes with the DiI-labeled MSCs. Scale bar 50 μm at ×4 mag, 20 μm at ×10 mag.

FIG. 3: Intravenously delivered, superparamagnetic iron oxide (SPIO)-loaded MSCs localise to lung metastases and can be visualized by MRI.

A) Representative coronal MRI sections (n=4 mice) of a normal mouse lung (Normal), mouse lung with metastases 35 days after intravenous delivery of MDAMB231 cells (pre MSC), and the same mouse lung one hour after SPIO-loaded MSC injection (post MSC). The metastases (circled) are visualized as focal regions of increased signal. These areas correspond to metastases on H&E histological sections (scale bar 100 μm). One hour post SPIO-loaded MSC injection, there is a decrease in signal intensity cause by the iron oxide in MSCs. (+ ribcage, * trachea, ̂ diaphragm with upper abdomen below, ˜ fissue separating lobes). B) The reduction in signal intensity secondary to the SPIO-loaded MSCs one hour and 24 hours post MSC injection was further confirmed and quantified by comparing signal-to-noise between the lung parenchyma and the deltoid muscle in three consecutive MR slices in 3 mice; there was a significant (p=0.005) reduction in signal-to-noise ratio across all 4 radiological areas (left upper (LU), lower (LL), right upper (RU), lower (RL). C) Tumour histology from mice harvested at day 35, one hour after SPIO-loaded MSC injection and MRI. i) Prussian blue and ii) DiI-staining (red) on contiguous sections from mice, demonstrating the MSCs migrate to and incorporate into lung metastases after intravenous delivery (scale bar 20 μm). iii) Macrophage immunohistochemistry (brown) stains different cells to SPIO-loaded cells (blue stain), iv) Macrophage immunofluorescence (green) stains different cells to DiI-labeled (red) cells (scale bar 5 μm).

### DETAILED DESCRIPTION OF THE INVENTION

### Tumours

Tumours that can be treated by the methods of the invention include primary tumours and metastatic tumours. Primary tumours that can be treated by the methods of the invention may include, but are not limited to, lung cancer, breast cancer, squamous cancer and cervical cancer. The methods of the invention can also be used to treat metastases in any part of the body. Metastases that can be treated by the methods of the invention include, but are not limited to, metastases that occur in the lung, liver, brain and bone. The methods of the invention are particularly useful for the treatment of pulmonary metastases. One preferred type of tumour to be treated is mesothelioma, a cancer that affects the pleural lining of the lung.

### Subjects

The methods of the invention can be used to treat a mammalian subject. Preferably, the methods of the invention are used to treat a human subject. Other mammals can also be treated, for example laboratory animals, such as mice, rats, rabbits and monkeys.

### Mesenchymal Stem Cells

The mesenchymal stem cells (MSCs) used in the invention can be obtained from any suitable source and are typically derived from the bone marrow, preferably human adult bone marrow. The MSCs used in the methods of the invention may be allogeneic (obtained from an individual other than the subject being treated) or syngeneic (obtained from the subject being treated). The MSCs used in the invention have the ability to migrate to and incorporate within the connective tissue stroma of tumours.

The MSCs of the invention are labelled with nanoparticles to enable the MSCs to be tracked.

### Delivery of MSCs

The MSCs of the invention can be delivered to the subject by either direct injection at the site of the tumour or by systemic delivery, for example by intravenous injection. Depending on the location and type of tumour, they can for example also be delivered intraperitoneally or (for example in the case of mesothelioma) to the pleural cavity.

### Homing

The term “homing” describes the ability of the MSCs of the invention to migrate to and incorporate within the connective tissue stroma of tumours.

### Vectors

In one embodiment of the invention, the pro-apoptotic factor is delivered to the tumour cells using a vector, preferably a viral vector. Viral vectors that can be used in the methods of the invention include, but are not limited to, adenoviral, lentiviral, adeno-associated viral (AAV), retroviral, mouse moloney leukaemia viral (MMLV), vaccinia viral or herpes simplex viral (HSV) vectors. Lentiviral vectors are the preferred vector for use in the present invention.

In one embodiment of the invention, the lentivirus has the ability to be conditionally activated. Preferably, the lentivirus is conditionally activated by a tetracycline, preferably a doxycycline. This inducible system allows a mixed cell and gene approach for metastatic cancers that can be activated and deactivated. Using this system, the MSCs can be infected at high efficiency.

### Pro-Apoptotic Factors

In one embodiment, the invention uses pro-apoptotic factors to kill the tumour cells. The pro-apoptotic factor used in the method of the invention will be capable of inducing the apoptosis pathway in a cell. Pro-apoptotic factors that can be used in the methods of the invention include, but are not limited to TRAIL, Bax, Bac, Fas receptor, caspase-3. Preferably the pro-apoptotic factor used for in the methods of the invention is TRAIL. TRAIL has been found to sensitise tumour cells both to chemotherapies and radiotherapies. Also, chemotherapies and radiotherapies have been found to sensitise tumour cells to TRAIL. Typically the pro-apoptotic factors are delivered to the tumour by incorporating the nucleic acid encoding the pro-apoptotic factor into a vector, preferably a viral vector.

### Nanoparticles

The nanoparticles of the invention may be metal or metal oxide nanoparticles and may for example contain cobalt, iron, cobalt and platinum or gold. The nanoparticles should be biocompatible or at least be of an acceptable level of toxicity at therapeutic dosage levels. Preferably, the nanoparticles used in the methods of the invention are iron oxide nanoparticles. In one embodiment of the invention, the nanoparticles are magnetic nanoparticles. Magnetic nanoparticles that can be used in the invention include ferromagnetic, ferromagnetic, or superparamagnetic nanoparticles. Preferably, the magnetic nanoparticles are superparamagnetic iron oxide (SPIO) nanoparticles.

### Imaging

The tracking of the MSCs homing towards a tumour is accomplished by imaging the nanoparticles that are contained within the MSCs. Any suitable imaging technique can be used to detect the nanoparticles contained within the MSCs. The imaging technique used should be capable of detecting the type of nanoparticles that are used in the method of the invention. Preferably, iron oxide nanoparticles are contained within the MSCs and MRI is used to detect the iron oxide nanoparticles. The types of MRI that may be used include T1 weighted scans, T2 weighted scans and T2* weighted scans. The MRI may be used to measure hypointensity and/or hyperintensity.

### Thermotherapy

In one embodiment of the invention, the magnetic nanoparticles contained within the MSCs are heated to kill tumour cells. This thermotherapy can be carried out using any of the nanoparticles discussed above using methods known in the art (see for example references 29-38). The heating of the nanoparticles is typically carried out by an alternating magnetic field (AMF) inducing inductor which is used to energize the nanoparticles. Preferably, the AMF inducing inductor is a resonant circuit device incorporated or embodied within an MRI apparatus. Alternatively, the AMF inducing device may be a separate apparatus.

### Chemotherapeutic Agents

In one embodiment of the invention, chemotherapeutic agents are used in conjunction with the MSC therapy as part of a combination therapy for the treatment of a tumour. The chemotherapeutic agents that can be used in the methods of the invention include, but are not limited to, alkylating agents, antimetabolites, anthracyclines, antitumour antibiotics, monoclonal antibodies, platinums, toopisomerases, tyrosine kinase inhibitors, plant alkaloids or histone deacetylase inhibitors

Antimetabolite chemotherapeutic agents that may be used in the methods of the present invention include, but are not limited to, Methotraxate, 6-mercaptopurine or 5-fluorouracil (5FU). Anthracycline chemotherapeutic agents that may be used in the present invention include, but are not limited to Daunorubicin, Doxorubicin, Idarubicin, Epirubicin, or Mitoxantrone. Antitumour antibiotic chemotherapeutic agents that may be used in the methods of the invention include, but are not limited to Bleomycin. Monoclonal antibody chemotherapeutic agents that may be used in the methods of the invention include, but are not limited to, Alemtuzumab (Campath), Bevacizumab (Avastin), Cetuximab (Erbitux), Gemtuzumab (Mylotarg), Ibritumomab (Zevalin), Panitumumab (Vectibix), Rituximab (Rituxan), Tositumomab (Bexxar), and Trastuzumab (Herceptin). Platinum chemotherapeutic agents that may be used in the methods of the invention include, but are not limited to, Cisplatin, Carboplatin or Oxaliplatin. Plant alkaloid chemotherapeutic agents that may be used in the methods of the invention include, but are not limited to toposiomerase inhibitors, Vinca alkaloids, taxanes such as paclitaxel or docetaxel, or Epipodophyllotoxins. Histone deacetylase inhibitors that may be used in the methods of the invention include, but are not limited to Vorinostat (suberoylanilide hydroxamic acid (SAHA), Zolinza), Romidepsin, Panobinostat, valproic acid, Belinostat, Mocetinostat, PCI-24781, Entinostat, SB939, Resminostat, Givinostat, CUDC-101, AR-42, CHR-2845, CHR-3996, 4SC-202 and sulforphane.

### Radiotherapy

In one embodiment of the invention, ionizing radiation or radiotherapy is used in conjunction with the MSC therapy as part of a combination therapy for the treatment of a tumour. The type of radiation that may be used in the methods of the invention include, but are not limited to, external beam radiotherapy (EBRT or XRT) or teletherapy, brachytherapy or sealed source radiotherapy, systemic radioisotope therapy or unsealed source radiotherapy.

### EXAMPLES

### Example 1

### Cell Culture

Tissue culture reagents were purchased from Invitrogen (Paisley, UK) unless otherwise stated. MDAMB231 breast cancer cells were obtained from Cancer Research UK, London Research Institute (CRUK, London, UK) and were cultured in DMEM and 10% fetal bovine serum (FBS). Human adult mesenchymal stem cells were purchased from Tulane University (New Orleans, USA) and cultured in αMEM with 16% FBS. FluidMAG iron nanoparticles (NC-D, Chemicell GmbH, Berlin, Germany) with a hydrodynamic diameter of 200 nm and a magnetite core were coated by the manufacturer with starch.

Labeling, phenotyping and visualisation of MSCs with iron nanoparticles

Labeling of MSCs with iron nanoparticles was performed by overnight incubation with 0.5 mg/ml nanoparticles in cell culture medium. The cells were vigorously washed with PBS 8 times to remove any free particles before use.

Adipogenic and osteogenic differentiation of MSCs was performed as previously described (11, 12). Cell viability was performed using an MTS NAD(P)H-dependent assay (13) according to the manufacturer's guidelines (Promega, Southampton UK). Cell apoptosis was analyzed using an Annexin-V-FITC/Propidium Iodide (A-V/PI) assay (ApoTarget™, Invitrogen), 72 hours after labeling. Ten samples were analyzed using a flow cytometer (FACSCalibur, Becton Dickenson, Oxford, UK), and 6×10³-8×10³ cells were scored per analysis (CellQuestPro, Becton Dickenson). Annexin V⁻PI⁻ cells were judged to be viable, Annexin V⁺/PI⁻ cells were considered to be undergoing apoptosis, and Annexin V⁺/PI⁺ cells were considered late apoptotic or necrotic, and recorded as dead (1).

Cell migration was performed as previously described (1). Briefly, 1.5×10⁵ MDAMB231 cells were plated in 800 μl medium on the bottom well of a transwell plate (Becton Dickenson), with 4×10⁴ MSCs in 300 μl plated in the upper well. The MSCs were allowed to migrate across the 8 μm pore membrane for 24 hours at 37° C. The cells attached to the upper side of the membrane were removed with a cotton bud, and the cells on the lower side that had migrated through the membrane were fixed, stained (Rapid Romanowsky, Raymond Lamb, Eastbourne, UK), and counted (5 fields/well, triplicate wells) at ×10 magnification (Olympus BX40, Watford, UK).

Prussian blue staining (1.2% potassium ferrocyanide with 1.8% hydrochloric acid) was performed on fixed cells (4% paraformaldehyde) 96 hours after labeling. Confocal microscopy was performed on a Leica TCS SP2 microscope (Leica Microsystems Ltd., Bucks., UK). Reflectance was used to visualize iron as previously described (14), and images were processed using Image J. For electron microscopy, cells were fixed with 2% paraformaldehyde, 1.5% glutaraldehyde in 0.1M phosphate buffer pH 7.3. They were then osmicated in 1% OSO4/0.1M phosphate buffer, dehydrated in a graded ethanol-water series, cleared in propylene oxide and infiltrated with Araldite resin. Ultra thin sections were cut using a diamond knife, collected on 300 mesh grids, then stained with uranyl acetate and lead citrate. These were viewed in a Jeol 1010 transmission electron microscope (Jeol, Herts., UK) and the images were recorded using a Gatan Orius CDD camera (Gatan, Abingdon, UK).

### Iron Quantification

We used a superconducting quantum interference device (SQUID) (15) to measure the amount of Fe₃O₄ in the cells. The samples were saturated in a field of 2 Tesla, which was subsequently removed to leave the superparamagnetic iron oxide (SPIO) particles in a magnetized state. Comparison of this remnant signal with a sample of known Fe₃O₄ concentration allowed quantification of Fe₃O₄ per cell.

### Xenograft Cancer Models

All animal studies were performed in accordance with British Home Office procedural and ethical guidelines. Six-week old NOD/SCID mice (Harlan, Bicester, UK) were kept in filter cages.

Subcutaneous tumors were obtained by the injection of 2×10⁶ MDAMB231 cells in 200 μl PBS, subcutaneously into the left flank with a 29 G needle. Metastatic lung tumors were produced by the intravenous delivery of two million MDAMB231 in 2000 PBS into the lateral tail vein.

### In Vivo Use of Iron Labeled-Mesenchymal Stem Cells

In the subcutaneous model, varying numbers of MSCs labeled with CM-DiI (Invitrogen, as per manufacturer's instructions), and iron nanoparticles were delivered concurrently with the cancer cells. In metastatic models, 7.5×10⁵ MSCs were suspended in 200 μl PBS and injected into the lateral tail vein at day 35 after the cancer models had been set up. As controls, MSCs not bearing nanoparticles, 100 ng of free iron, or iron nanoparticle-labeled MSCs which were killed in 70% ethanol (cell death confirmed with trypan blue staining) were delivered with the cancer cells.

### Magnetic Resonance Imaging (MRI)

Images were acquired on a 9.4T horizontal bore Varian (VNMRS) system using a 39 mm RF coil (RAPID Biomedical GmbH). Lung in vivo images were obtained before, one hour and 24 hours after MSC injection, at day 35 after the metastatic model had been initiated (n=4 mice). They were acquired using a fast spin-echo sequence with cardiac and respiratory gating (TR˜1 s, effective TE=5 ms, 100 um in-plane resolution, 1 mm slice thickness, NSA=4). Subcutaneous tumor images were obtained 28 days after subcutaneous injection of MDAMB231 cells and MSCs and acquired ex vivo using the same sequence and similar parameters (TR=1.5 s, effective TE=5 ms, 100 um in-plane resolution, 1 mm slice thickness, NSA=4) (n=14 mice; 2 per group). Signal-to-noise ratios were obtained from three consecutive coronal slices for 4 lung areas (right and left, upper and lower), using the average signal intensity (SI) of each area, the SI of shoulder muscle and the standard deviation of the noise, within each slice.

### Immunohistochemistry

Mice were sacrificed by CO₂ asphyxiation followed by exsanguination following the MRI at day 28 in the subcutaneous tumour experiment and post final MRI (1 hour or 24 hours following MSC delivery) in the metastatic experiment. Subcutaneous tumors were removed and fixed in 4% paraformaldehyde for histology. The lungs were excised and inflated with a fixed 20 cm pressure of 4% paraformaldehyde and then bathed in 4% paraformaldehyde for histology.

Fixed specimens were embedded in paraffin and cut into 3 μm sections for Haematoxylin and Eosin (H&E) staining. Prussian Blue staining was used to detect iron and fluorescent microscopy was used to detect DiI positive cells with DAPI counterstain. Macrophages were stained with a monoclonal rat anti-mouse Mac-2 primary antibody (1/10000 dilution, Cedarlane, Ontario, Canada) for immunohistochemistry and a monoclonal rat anti-mouse F4/80 primary antibody (1/50 dilution, Ebiosciences, Herts., UK) for immunofluorescence. Microscopy was performed using light (Olympus BX40) or fluorescent (Carl Zeiss Ltd., Axioskop 2, Welwyn Garden City, UK) microscopes.

### Statistics

Statistical analysis was performed using GraphPad Prism v4 (GraphPad Software, California, USA). Multiple groups were analysed by Anova. Single group data was assessed using Student's t-test or Mann-Whitney test. Results were considered to be statistically significant for p<0.05.

### Results

### Iron Labeling of MSCs

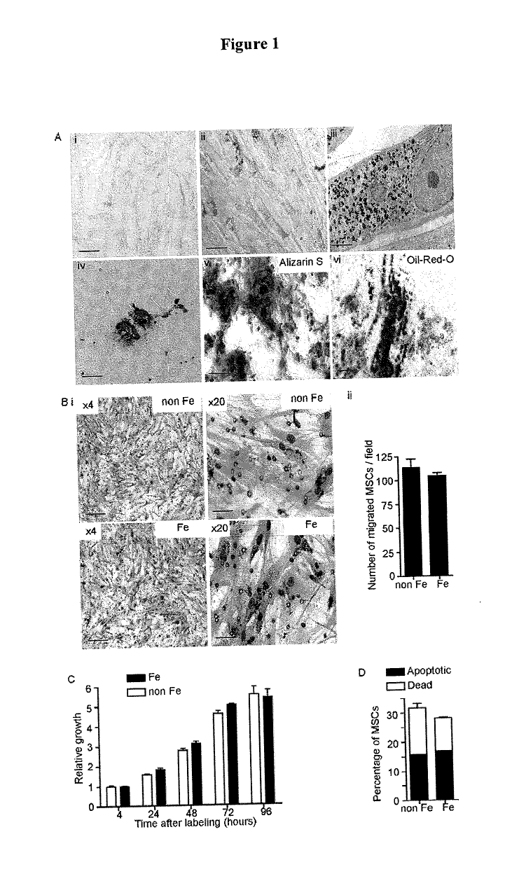

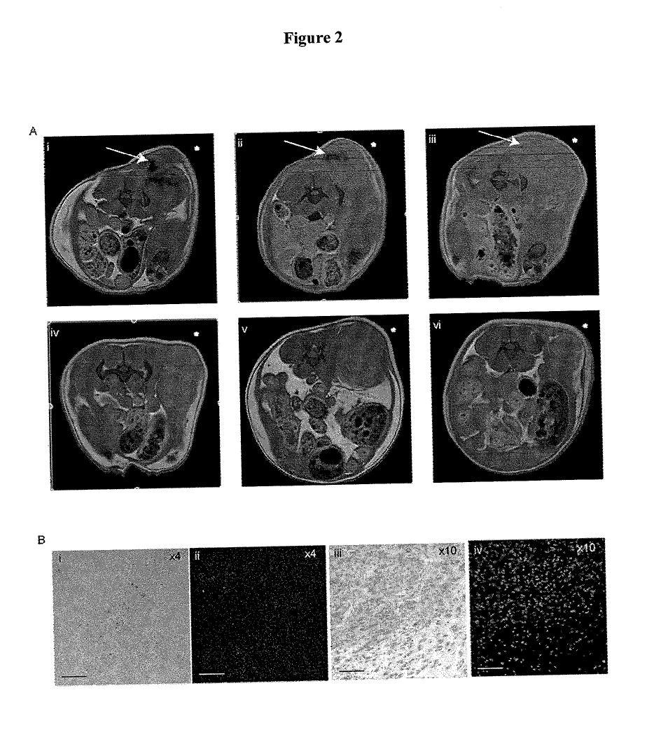

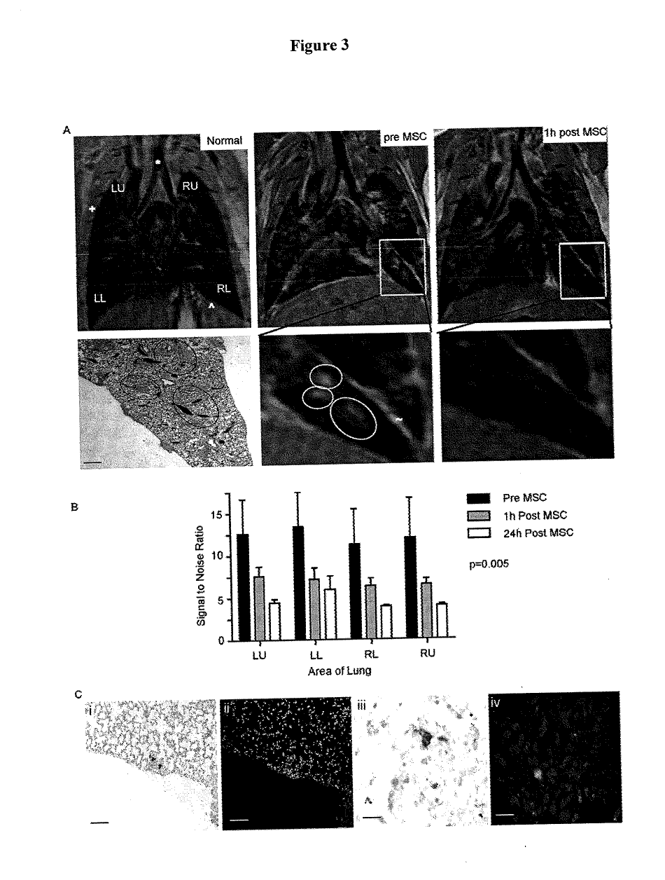

The MSCs readily internalized the iron nanoparticles. This was confirmed by Prussian blue staining, electron microscopy and confocal microscopy (FIG. 1Ai-iv). Cells contained up to 30 pg of iron oxide per MSC, quantified using SQUID magnetometry. The labeled cells retained their MSC characteristics, with the ability to differentiate into stromal tissues, including bone and fat (FIG. 1Av-vi). Furthermore, the iron nanoparticle-labeled and unlabeled MSCs demonstrated equivalent in vitro tumor homing (104.4±5.6 vs. 113.1±16.1 cells/field) in transwell migration studies (non-significant (ns), t-test) (FIG. 1B). There was also no effect of iron nanoparticles on MSC proliferation (ns, 2-way Anova), as demonstrated by the MTS proliferation assay (FIG. 1C), or cell viability as demonstrated by Annexin V flow cytometry apoptosis assay (33.0±4.2% cells dead or apoptotic cells with no iron nanoparticles, compared to 29.4±2.1% with iron nanoparticles, ns, Mann-Whitney) (FIG. 1D).

### Detection and Sensitivity of MRI to Iron Labeled Cells

To determine the sensitivity of MRI in visualizing MSCs carrying iron nanoparticles, we used subcutaneous tumors, rather than lung tissue, in combination with our lung imaging MRI sequence to assess the dose response of iron labeled cells, as the air spaces in the lung could confound this assessment. We grew subcutaneous MDAMB231 tumors (2×10⁶ cells) in NOD/SCID mice with increasing numbers of DiI-labeled human MSCs carrying nanoparticles (100, 1×10³, 1×10⁴, and 1×10⁵) for 28 days (n=2 per group). Using a 9.4T MRI system we were able to visualize as few as 1000 MSCs labeled with nanoparticles in tumours 28 days after injection of the MDAMB231 cells (FIG. 2Ai-iii). Signal voids were not visible at 28 days when non-iron-labeled MSCs, dead iron-labeled MSCs (FIG. 2Av), or free iron (FIG. 2A vi) were coadministered with the tumour cells. Histopathological examination confirmed that iron was present only in the tumors injected with live nanoparticle-labeled MSCs. This was demonstrated by co-localization of the Prussian blue staining of iron and DiI fluorescence with the MSCs (FIG. 2B).

### Homing and In Vivo Detection of Iron-Labeled MSCs to Lung Tumors

In the following experiments, 2×10⁶ MDAB231 cells were injected into the tail vein. This model reproducibly forms pulmonary metastases throughout all lung lobes. We were able to detect lung metastases using MRI, visualized as diffuse hyperintensities in all five lobes 38 days after tumour cell injection (FIG. 3A). As we have shown previously, MSCs show tropism to pulmonary tumors (1). Therefore 35 days post intra-venous delivery of MDAMB231 cells, MSCs double-labeled with DiI and iron nanoparticles were injected intravenously. We used MRI to confirm the fate of the intravenously injected, iron-labeled MSCs within metastases in vivo, by acquiring MR images pre-, one hour, and 24 hours post-injection. MRI images post MSC injection showed a decrease in signal intensity in areas of metastatic deposits detected in pre-MSC delivery images, which correlated with the iron-labeled MSCs integrating or lodging into these tumors (n=4) (FIG. 3A). To examine MSC engraftment throughout the lung, the signal intensity across the lung was examined before and after MSC injection in three consecutive slices. This was compared to the standard deviation of the signal noise of each slice, giving a within-slice signal-to-noise ratio (SNR) for each examined area, which was averaged across the three slices. There was a significant reduction in the SNR following the MSC injection, which was consistent in all lung areas (p=0.005, 2-way Anova, n=3) (FIG. 3B). There were no differences in the SNR decrease between the lung areas (ns, 2-way Anova). Immunohistochemistry confirmed our previous findings that DiI/Fe staining cells were found within or adjacent to tumors (FIG. 3Ci,ii). As previous studies have suggested that iron-labeled cells may represent macrophages, we performed immunohistochemistry and immunofluorescence for macrophages and iron or DiI (16, 17). There was no colocalisation of the macrophage marker with the iron nanoparticles or DiI-positive cells with either technique (FIG. 3C iii-iv).

### REFERENCES

1. Loebinger M R et al. Mesenchymal stem cells reduce lung metastases by controlled targeted delivery of tumour necrosis factor-related apoptosis-inducing ligand. Thorax 2008; 63(Suppl VII): A1-A3.
2. Kim S M, Lim J Y, Park S I, et al. Gene therapy using TRAIL-secreting human umbilical cord blood-derived mesenchymal stem cells against intracranial glioma. Cancer Res 2008; 68:9614-23.
3. Nakamizo A, Marini F, Amano T, et al. Human bone marrow-derived mesenchymal stem cells in the treatment of gliomas. Cancer Res 2005; 65:3307-18.
4. Studeny M, Marini F C, Champlin R E, Zompetta C, Fidler I J, Andreeff M. Bone marrow-derived mesenchymal stem cells as vehicles for interferon-β delivery into tumors. Cancer Res 2002; 62:3603-8.
5. Studeny M, Marini F C, Dembinski J L, et al. Mesenchymal stem cells: potential precursors for tumor stroma and targeted-delivery vehicles for anticancer agents. Natl Cancer Inst 2004; 96:1593-603.
6. Xin H, Kanehira M, Mizuguchi H, et al. Targeted delivery of CX3CL1 to multiple lung tumors by mesenchymal stem cells. Stem Cells 2007; 25:1618-26.
7. Marx J C, Allay J A, Persons D A, et al. High-efficiency transduction and long-term gene expression with a murine stem cell retroviral vector encoding the green fluorescence protein in human marrow stromal cells. Hum Gene Ther 1999; 10:1163-73.
8. Le Blanc K, Tammik L, Sundberg B, Haynesworth S E, Ringden O. Mesenchymal stem cells inhibit and stimulate mixed lymphocyte cultures and mitogenic responses independently of the major histocompatibility complex. Scand J Immunol 2003; 57:11-20.
9. Javazon E H, Beggs K J, Flake A W. Mesenchymal stem cells: paradoxes of passaging. Exp Hematol 2004; 32:414-25.
10. Bulte J W, Kraitchman D L. Iron oxide MR contrast agents for molecular and cellular imaging. NMR Biomed 2004; 17:484-99.
11. Kyrtatos P G, Lehtolainen P, Junemann-Ramirez M, et al. Magnetic tagging increases delivery of circulating progenitors in vascular injury. JACC Cardiovasc Interv 2009; 2:794-802.
12. Panizzo R A, Kyrtatos P G, Price A N, Gadian D G, Ferretti P, Lythgoe M F. In vivo magnetic resonance imaging of endogenous neuroblasts labelled with a ferumoxide-polycation complex. Neuroimage 2009; 44: 1239-46.
13. Aguilar S, Nye E, Chan J, et al. Murine but not human mesenchymal stem cells generate osteosarcoma-like lesions in the lung. Stem Cells 2007; 25: 1586-94.
14. Chan J, O'Donoghue K, de la Fuente J, et al. Human fetal mesenchymal stem cells as vehicles for gene delivery. Stem Cells 2005; 23:93-102.
15. Berridge M V, Tan A S. Characterization of the cellular reduction of 3-(4,5-dimethylthiazol-2-yl)-2,5-diphenyltetrazolium bromide (MTT): subcellular localization, substrate dependence, and involvement of mitochondrial electron transport in MTT reduction. Arch Biochem Biophys 1993; 303:474-82.
16. Hautot D, Pankhurst Q A, Dobson J. Superconducting quantum interference device measurements of dilute magnetic materials in biological samples. Rev Sci Instrum 2005; 76:45101.
17. Loebinger M R, Giangreco A, Groot K R, et al. Squamous cell cancers contain a side population of stem-like cells that are made chemosensitive by ABC transporter blockade. Br J Cancer 2008; 98:380-7.
18. Amsalem Y, Mardor Y, Feinberg M S, et al. Iron-oxide labeling and outcome of transplanted mesenchymal stem cells in the infarcted myocardium. Circulation 2007; 116:138-45.
19. Terrovitis J, Stuber M, Youssef A, et al. Magnetic resonance imaging overestimates ferumoxide-labeled stem cell survival after transplantation in the heart. Circulation 2008; 117:1555-62.
20. Fox J M, Chamberlain G, Ashton B A, Middleton J. Recent advances into the understanding of mesenchymal stem cell trafficking. Br J Haematol 2007; 137:491-502.
21. Loebinger M R, Sage E K, Janes S M. Mesenchymal stem cells as vectors for lung disease. Proc Am Thorac Soc 2008; 5:711-6.
22. Klopp A H, Spaeth E L, Dembinski J L, et al. Tumor irradiation increases the recruitment of circulating mesenchymal stem cells into the tumor microenvironment. Cancer Res 2007; 67:11687-95.
23. Hung S C, Deng W P, Yang W K, et al. Mesenchymal stem cell targeting of microscopic tumors and tumor stroma development monitored by noninvasive in vivo positron emission tomography imaging. Clin Cancer Res 2005; 11:7749-56.
24. Delcroix G J, Jacquart M, Lemaire L, et al. Mesenchymal and neural stem cells labeled with HEDP-coated SPIO nanoparticles: in vitro characterization and migration potential in rat brain. Brain Res 2009; 1255:18-31.
25. Khakoo A Y, Pati S, Anderson S A, et al. Human mesenchymal stem cells exert potent antitumorigenic effects in a model of Kaposi's sarcoma. J Exp Med 2006; 203:1235-47.
26. Mohr A et al. Mesenchymal stem cells expressing TRAIL lead to tumour growth and inhibition in an experimental lung cancer model. J Cell Mol Med 2008; 12: 2628-2643.
27. Sasportas L S et al. Assessment of therapeutic efficacy and fate of engineered human mesenchymal stem cells for cancer therapy. PNAS 2009: 106:4822-4827.
28. Menon L G et al. Human bone marrow derived mesenchymal stromal cells expressing S-TRAIL as a cellular delivery vehicle for human glioma therapy. Stem Cells 2009:27:2320-2330.
29. WO 2009/076673 A2.
30. US 2005/090732.
31. U.S. Pat. No. 4,983,159.
32. U.S. Pat. No. 4,369,345.
33. JP 3128331.
34. WO 9107132.
35. U.S. Pat. No. 5,411,730.
36. WO 9725062.
37. U.S. Pat. No. 6,149,576.
38. FR 2848850.
39. WO 2005/059118.

1. A method of treating a tumour in a subject comprising the steps of:
delivering mesenchymal stem cells (MSCs) labelled with metal or metal oxide nanoparticles;
using imaging to detect homing of said MSCs containing said nanoparticles towards the cells of the tumour; and
killing said tumour cells by delivery of a pro-apoptotic factor by said MSCs to said tumour cells.

2. The method of claim 1 wherein the nanoparticles are magnetic iron oxide nanoparticles and detection of the homing of said MSCs is carried out using magnetic resonance imaging (MRI).

3. The method of claim 1 wherein said MSCs are delivered directly to said tumour.

4. The method of claim 1 wherein said MSCs are delivered systemically by intravenous injection.

5. The method of claim 1 wherein said tumour is a primary tumour.

6. The method of claim 1 wherein said tumour is a metastasis.

7. The method of claim 1 wherein said tumour is a pulmonary metastasis.

8. The method of claim 7 wherein said pulmonary metastasis originates from a breast, lung, squamous, cervical, gastrointestinal, kidney, melanoma, sarcomas, lymphomas, testicular or leukaemia primary tumour.

9. The method of claim 1 wherein said pro-apoptototic factor is encoded by a nucleic acid transduced into said MSCs.

10. The method of claim 1 wherein said pro-apoptotic factor is delivered by viral expression.

11. The method of claim 10 wherein said pro-apoptotic factor is virally expressed from a lentiviral vector.

12. The method of claim 1 wherein said pro-apoptotic factor is tumour necrosis factor-related apoptosis-inducing ligand (TRAIL).

13. The method of claim 1 wherein said MSCs are from the same species as the subject.

14. The method of claim 13 wherein said MSCs are human adult MSCs and said subject is a human subject.

15. The method of claim 1 wherein said delivery of said MSCs is carried out in combination with treatment of said tumour with an anti-tumour chemotherapeutic agent.

16. The method of claim 1 wherein said delivery of said MSCs is carried out in combination with treatment of said tumour with ionizing radiation.

17. The method of claim 2 wherein delivery of said pro-apoptotic agent is carried out in combination with the killing of said tumour cells by thermotherapy.

18. A method of treating a tumour in a subject comprising the steps of:
delivering MSCs labelled with magnetic nanoparticles;
detecting homing of said MSCs towards the cells of the tumour using MRI; and
killing said tumour cells by thermotherapy.

19. The method of claim 18 wherein said magnetic nanoparticles are superparamagnetic iron oxide nanoparticles.

20. The method of claim 18 wherein said MSCs are delivered directly to said tumour.

21. The method of claim 18 wherein said MSCs are delivered systemically by intravenous injection.

22. The method of claim 18 wherein said tumour is a primary tumour.

23. The method of claim 18 wherein said tumour is a metastasis.

24. The method of claim 18 wherein said tumour is a pulmonary metastasis.

25. The method of claim 24 wherein said pulmonary metastasis originates from a breast, lung, squamous, cervical, gastrointestinal, kidney, melanoma, sarcomas, lymphomas, testicular or leukaemia primary tumour.

26. The method of claim 18 wherein said MSCs are from the same species as the subject.

27. The method of claim 18 wherein said MSCs are human adult MSCs and said subject is a human subject.

28. The method of claim 18 wherein the thermotherapy is carried out by an alternating magnetic field (AMF) inducing inductor.

29. The method of claim 18 in which said delivery of said MSCs is carried out in combination with treatment with an anti-tumour chemotherapeutic agent.

30. The method of claim 18 wherein said delivery of said MSCs is carried out in combination with treatment of said tumour with ionizing radiation.

31. The method of claim 18 wherein the thermotherapy is carried out in combination with the delivery of a pro-apoptotic agent by said MSCs.

32. A method of treating pulmonary metastases comprising the steps of:
systemically delivering MSCs labelled with superparamagnetic iron oxide nanoparticles;
detecting homing of said MSCs towards the cells of said pulmonary metastases using MRI; and
killing said tumour cells by delivery of TRAIL to said tumour cells using a lentiviral vector within said MSCs.

33. A method of treating pulmonary metastases comprising the steps of:
systemically delivering MSCs labelled with superparamagnetic iron oxide nanoparticles;
detecting homing of said MSCs towards the cells of said pulmonary metastases using MRI; and
killing said tumour cells by thermotherapy.

34. The method of claim 1 wherein said MSCs are delivered intraperitoneally or to the pleural cavity.

35. The method of claim 1 wherein the tumour to be treated is a mesothelioma and said MSCs are delivered to the pleural cavity.

36. The method of claim 18 wherein said MSCs are delivered intraperitoneally or to the pleural cavity.

37. The method of claim 18 wherein the tumour to be treated is a mesothelioma and said MSCs are delivered to the pleural cavity.
